(19) United States
(12) Patent Application Publication
(10) Pub. No.: US 2025/0237636 A1
(43) Pub. Date: Jul. 24, 2025

(54) MILK MEASURING DEVICE

(71) Applicant: COWEALTHY TEKNOLOJI ANONIM SIRKETI, Gebze, Kocaeli (TR)

(72) Inventors: Yucel SAYKAL, Gebze, Kocaeli (TR); Ismail BICAN, Gebze, Kocaeli (TR); Anil SIRMA, Gebze, Kocaeli (TR); Enes OZKAYA, Gebze, Kocaeli (TR); Ibrahim Ebuzer YAYLA, Gebze, Kocaeli (TR); Mustafa Kemal UYGUN, Gebze, Kocaeli (TR)

(21) Appl. No.: 18/855,497

(22) PCT Filed: Feb. 14, 2023
(86) PCT No.: PCT/TR2023/050141

§ 371 Date: Oct. 9, 2024

(30) Foreign Application Priority Data
Apr. 28, 2022 (TR) 2022/007049

(51) Int. Cl. G01N 33/04; A01J 5/01; A01J 5/013; G01F 1/661; G01N 21/3577

(52) U.S. Cl. CPC G01N 33/04; A01J 5/01; A01J 5/0133; A01J 5/0135; G01F 1/661; G01N 21/3577

(57) ABSTRACT

Disclosed is a milk measuring device for determining at least one parameter of milk flowing through a flow line provided between an inlet opening and an outlet opening. The flow line has a first circuit board and a second circuit board. The first circuit board has a plurality of infrared transmitters directed towards said flow line for emitting infrared rays. The second circuit board has a plurality of infrared receivers directed towards said first circuit board for detecting the infrared rays emitted and generating a signal according to the detected infrared rays. The infrared rays emitted are detected by at least two infrared receivers having a processor unit for ensuring simultaneous operation of the infrared transmitters and infrared receivers. The processor unit is configured to determine at least one flow parameter of the milk flowing through the flow line according to the signal generated by the infrared receivers.

### TECHNICAL FIELD

The invention relates to a milk measuring device for determining at least one parameter of milk flowing through a flow line provided between an inlet opening and an outlet opening.

### BACKGROUND

Milk measuring devices are used to determine certain parameters of milk milked in milking parlors. These parameters can be mastitis, blood, or foreign matter content in the milk. These parameters provide very important information to dairy farm owners about the quality of milk. Because the introduction of milk of inadequate quality to the market poses a threat to the health of consumers and may cause criminal proceedings for dairy farm owners.

The said parameters are usually realized in a warehouse outside the milking household after milking. Different devices are used for each parameter to obtain information about milk quality. The inability to measure milk quality during milking adds workload after milking. Moreover, milk of poor quality or milk containing impurities such as blood, mastitis, etc. requires milk to be stored in containers associated with each animal in order to know from which animal it was milked. This results in additional labor and storage costs.

As a result, all the above-mentioned problems have made it imperative to innovate in the related technical field.

### BRIEF DESCRIPTION OF THE INVENTION

The present invention relates to a milk measuring device for eliminating the aforementioned disadvantages and bringing new advantages to the related technical field.

One object of the invention is to provide a milk measuring device for monitoring the quality of milk in real-time during milking.

A further object of the invention is to provide a milk measuring device for determining the amount of milk milked during milking with improved accuracy.

A further object of the invention is to provide a milk measuring device for monitoring the quality of milk and the animal in real-time during milking.

In order to fulfill all the aforementioned objects and those which will arise from the following detailed description, the present invention relates to a milk measuring device for determining at least one parameter of milk flowing through a flow line provided between an inlet opening and an outlet opening. Accordingly, it is characterized in that; the said flow line comprises a first circuit board and a second circuit board configured to remain therebetween; said first circuit board comprises a plurality of infrared transmitters directed towards said flow line for emitting infrared rays; said second circuit board comprises a plurality of infrared receivers directed towards said first circuit board for detecting the infrared ray emitted by said infrared transmitters and generating a signal according to the detected infrared ray; the infrared ray emitted by each infrared transmitter is detected by at least two infrared receivers; that it comprises a processor unit for ensuring simultaneous operation of the infrared transmitter and the infrared receivers, and the processor unit is configured to determine at least one flow parameter of the milk flowing through the flow line according to the signal generated by the infrared receivers. This improves the measurement accuracy of the amount of milk flowing through the flow line. The milk measuring device is able to determine the amount of milk flowing through the flow line in three dimensions. At the same time, the amount of air flowing through the flow line can also be determined.

A possible embodiment of the invention is characterized in that the infrared transmitter provided on the first circuit board and the infrared receivers provided on the second circuit board are arranged in equal numbers and opposite each other. This ensures that the infrared ray emitted from the infrared transmitter is transmitted perpendicularly to at least one infrared receiver.

Another possible embodiment of the invention is characterized in that the infrared transmitters provided on the first circuit board and the infrared receivers provided on the second circuit board are arranged in two rows of five. Thus, the infrared rays emitted by the infrared transmitters are detected by the infrared receivers at a right angle, at a narrower angle than the right angle, and at an even narrower angle than the narrow angle. Thus, the quantities of milk and air flowing through the flow line are determined with improved accuracy.

Another possible embodiment of the invention is characterized in that the infrared ray emitted by each infrared transmitter is detected by the infrared receiver provided opposite thereto and at least five infrared receivers closest to the infrared receiver provided opposite thereto. This allows for the determination of the amount of milk flowing through the flow line with increased accuracy.

Another possible embodiment of the invention is characterized in that it comprises a conductivity sensor for measuring the conductivity of milk, configured to be at least partially in contact with the milk flowing through the flow line, the processor unit is configured to determine at least one conductivity parameter of the milk flowing through the flow line according to the signal it receives from said conductivity sensor. Thus, the electrical conductivity of the milk flowing through the flow line is determined by the processor unit.

Another possible embodiment of the invention is characterized in that it comprises a heat sensor for measuring the temperature of milk flowing through the flow line, the processor unit is configured to determine at least one heat parameter of milk flowing through the flow line according to the signal it receives from said heat sensor. Thus, the temperature of the milk flowing through the flow line is determined. In addition, inflammation, burning, infection, inflammation, or any problem in the breast can be detected according to the temperature and conductivity change.

Another possible embodiment of the invention is characterized in that the first circuit board comprises a light source directed towards the flow line for emitting visible light wavelength; the second circuit board comprises an optical sensor directed towards the first circuit board for detecting the light emitted by said light source and generating a signal according to the light detected; the processor unit is configured to determine at least one color parameter of the milk flowing through the flow line according to the signal it receives from the said optical sensor. Thus, the color analysis of the milk flowing through the flow line is performed. The light transmittance of the milk flowing through the flow line is measured according to different wavelengths.

Another possible embodiment of the invention is characterized in that it comprises a pressure sensor for measuring the pressure of the milk flowing through the flow line and a pressure transmission element for providing a relation between the said pressure sensor and the milk flowing through the flow line; the processor unit is configured to determine at least one pressure parameter of the milk flowing through the flow line according to the pressure signal it receives from the pressure sensor. Thus, the vacuum and pulsation test of the milking system is performed by means of the pressure measurement information. With the vacuum and pulsation test, problems in the milking system are detected. In addition, pressure information is also used in milk measuring device measurement calibration.

Another possible embodiment of the invention is characterized in that said flow line is made of infrared ray transmitting material. Thus, the infrared ray emitted by the infrared data passes through the flow line and is detected by the infrared receiver.

Another possible embodiment of the invention is characterized in that at least part of said flow line is configured to pass visible light wavelength. Thus, the light emitted by the light source can pass through the flow line and be detected by the optical sensor.

Another possible embodiment of the invention is characterized in that it comprises a memory unit; the processor unit is configured to store the determined parameters in said memory unit by associating it with the milked animal information. Thus, data from animals in the same group with common parameters such as breed, weight, height, food eaten, same lactation periods can be collected in a data pool and compared with each other. In this way, an assumption can be made using machine learning, artificial intelligence algorithms, and similar tools.

Another possible embodiment of the invention is characterized in that it comprises a communication unit for enabling external industrial devices to communicate with the milk measuring device; said communication unit is associated with the processor unit. Thus, the milk measuring device is enabled to exchange data with external devices.

Another possible embodiment of the invention is characterized in that it comprises a user interface to enable the user to read the parameters determined by the processor unit. Thus, the parameters determined by the milk measuring device are transmitted to the user. The user can also read old data stored in the memory unit through the user interface. When the animal comes to the milking parlor, the milk measuring device recognizes which animal this animal is, so it can make comments about its health by comparing the current and old data through the memory unit. Recognition of this animal can be done by a handheld terminal connected through the communication unit or by electronic data entry through the user interface. The milk measuring device displays not only the current amount of milk flowing but also the previous data of the animal from the memory unit and displays this data on the user interface. The user interface also allows the monitoring of the data of all animals on a single screen.

### BRIEF DESCRIPTION OF THE FIGURE

FIG. 1 shows a representative exploded view of the milk measuring device.

FIG. 2 shows a representative view of the first circuit board and the second circuit board.

FIG. 3 shows a schematic view of the milk measuring device.

FIG. 4 shows a representative view of a preferred embodiment of a milk measuring device.

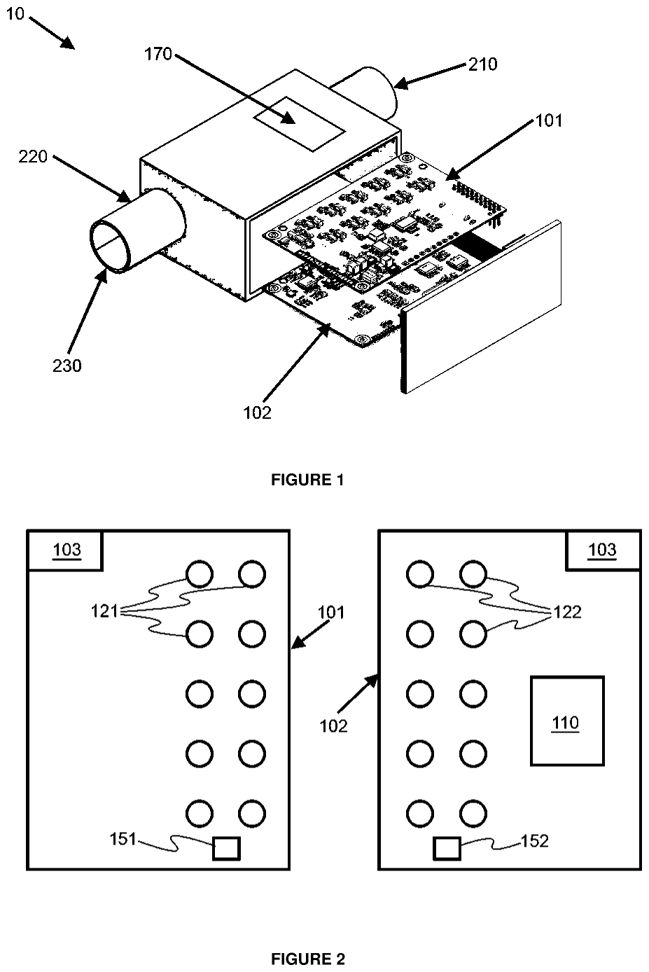

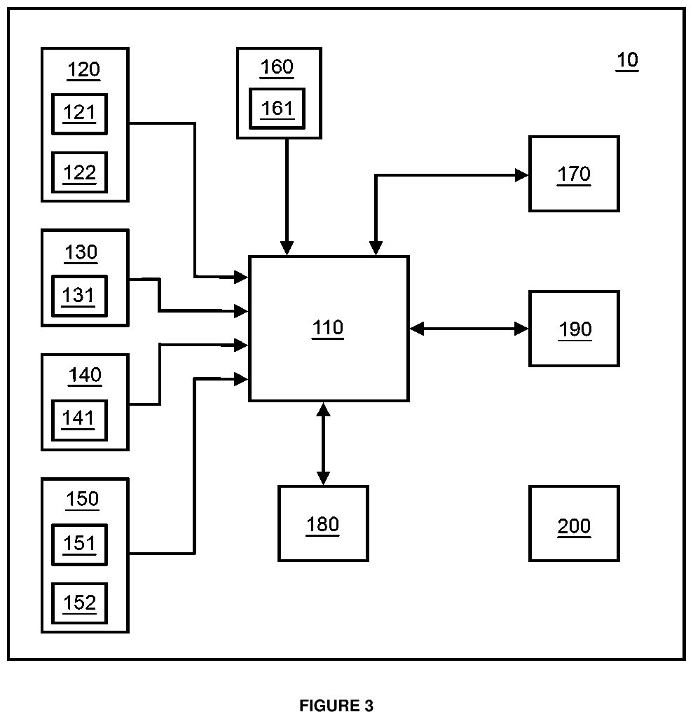

### DETAILED DESCRIPTION OF THE INVENTION

In this detailed description, the milk measuring device (10) of the invention is described only by way of non-limiting examples for a better understanding of the subject matter.

One of the most important criteria in industrial dairy farms is milk quality. Milk quality and milk quantity also provide information on animal health. For this reason, it is particularly important to instantaneously measure the milk milked from the animals separately for each animal during milking. The object of the invention is to provide a milk measuring device (10) for measuring the milk quantity and milk quality of each animal in real time without touching the milk during milking in dairy farms. The quality of the milk mentioned herein is determined by detecting impurities in the milk such as mastitis, blood, and fat. The quantity of said milk is determined by determining the volume of milk that is milked. The milk measuring device (10) subject to the invention also enables monitoring of the yield of each animal according to the quality and quantity of milk milked from the animals.

The milk measuring device comprises an inlet opening (210) for the inlet of milk and an outlet opening (230) for the outlet of milk. The milk measuring device (10) comprises a flow line (220) provided between said inlet opening (210) and said outlet opening (230). The milked milk flows along said flow line (220) from said inlet opening (210) towards said outlet opening (230). The milk measuring device (10) determines at least one parameter of the milk flowing through the flow line (220). In a possible embodiment, the flow line (220) is made of an infrared ray transmitting material. In a possible embodiment, at least a region of the flow line (220) is configured to transmit visible light wavelength.

The milk measuring device comprises a processor unit (110) for determining at least one parameter of milk flowing through the flow line (220). The milk measuring device (10) further comprises a first circuit board (101) and a second circuit board (102). The said first circuit board (101) and said second circuit board (102) are provided opposite each other and with a flow line (220) between them. Said processor unit (110) may be positioned on either the first circuit board (101) or the second circuit board (102). The first circuit board (101) and the second circuit board (102) may be any electronic circuit board or printed circuit board known in the art. In the preferred embodiment, the first circuit board (101) and the second circuit board (102) are double-layer PCB circuit boards.

The first circuit board comprises a plurality of infrared transmitters (121) directed towards the flow line (220) for emitting an infrared ray. The second circuit board (102) comprises a plurality of infrared receivers (122) directed towards the first circuit board (101) for detecting the infrared ray emitted by said infrared transmitters (121). Said infrared receivers (122) generate a signal according to the detected infrared ray. The processor unit (110) ensures the simultaneous operation of the infrared transmitter (121) and the infrared receivers (122). The processor unit (110) comprises a flow measurement unit (120). Said flow measurement unit (120) is configured to determine at least one flow parameter of the milk flowing through the flow line (220) according to the signal generated by the infrared receivers. The flow measurement unit (120) determines the flow rate and/or volume of milk flowing through the flow line (220). The flow measurement unit (120) determines the flow parameter of the milk flowing through the flow line (220) by detecting the infrared ray emitted by each infrared emitter (121) by at least two infrared receivers (122). Thus, the amount of milk flowing through the flow line (220) is determined with improved accuracy.

In a possible embodiment of the invention, the infrared transmitters (121) provided on the first circuit board (101) and the infrared receivers (122) provided on the second circuit board (102) are arranged in equal numbers and opposite each other. In a possible embodiment, the infrared transmitters (121) and infrared receivers (122) are provided in two rows of five. In the preferred embodiment of the invention, the infrared ray emitted by each infrared transmitter (121) is detected by the infrared receiver (122) provided opposite thereto and at least five infrared receivers (122) closest to the infrared receiver (122) provided opposite thereto. In this way, the accuracy of the amount of milk flowing through the flow line (220) determined by the flow measurement unit (120) is further increased.

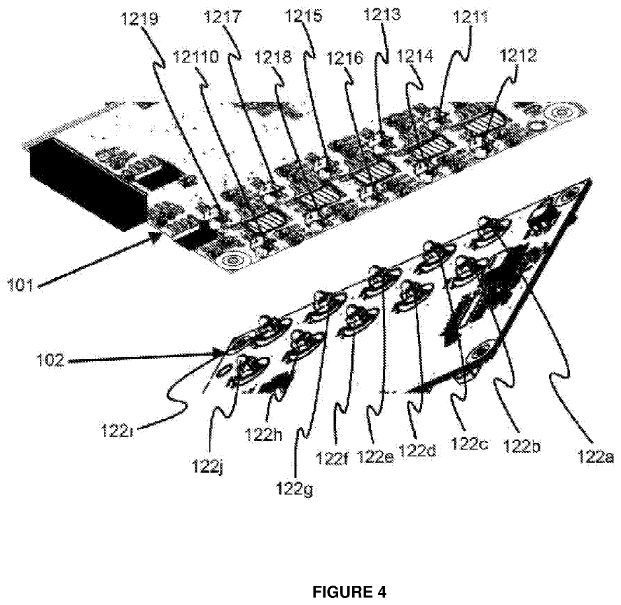

Referring to FIG. 4, in the preferred embodiment of the invention, the infrared transmitters (121) are designated from 1211 to 12110 and the infrared receivers (122) are designated from 122a to 122j. In this embodiment;
1211. the infrared ray emitted by the infrared transmitter is detected by 122a-b-c-d-e and f infrared receivers,
1212. the infrared ray emitted by the infrared transmitter is detected by 122a-b-c-d-e and f infrared receivers,
1213. the infrared ray emitted by the infrared transmitter is detected by 122a-b-c-d-e-f-g and h infrared receivers,
1214. the infrared ray emitted by the infrared transmitter is detected by 122a-b-c-d-e-f-g and h infrared receivers,
1215. the infrared ray emitted by the infrared transmitter is detected by 122a-b-c-d-e-f-g-h-i and j infrared receivers,
1216. the infrared ray emitted by the infrared transmitter is detected by 122a-b-c-d-e-f-g-h-i and j infrared receivers,
1217. the infrared ray emitted by the infrared transmitter is detected by 122c-d-e-f-g-h-i and j infrared receivers,
1218. the infrared ray emitted by the infrared transmitter (121) is detected by 122c-d-e-f-g-h-i and j infrared receivers,
1219. the infrared ray emitted by the infrared transmitter (121) is detected by 122e-f-g-h-i and j infrared receivers,
12110. the infrared ray emitted by the infrared transmitter (121) is detected by 122e-f-g-h-i and j infrared receivers. Therefore, the infrared ray emitted by 1211. and 1212. infrared transmitters are detected by at least 12 infrared receivers (122), including at least 6 infrared receivers; the infrared ray emitted by 1213. and 1214. infrared transmitters are detected by at least 16 infrared receivers (122), including at least 8 infrared receivers; the infrared ray emitted by 1215. and 1216. infrared transmitters are detected by at least 20 infrared receivers (122), including at least 10 infrared receivers; the infrared ray emitted by 1217. and 1218. infrared transmitters are detected by at least 16 infrared receivers (122), including at least 8 infrared receivers; the infrared ray emitted by 1219. and 12110. infrared transmitters are detected by at least 12 infrared receivers (122), including at least 6 infrared receivers. This detection operation, which occurs in 10 thousandths of a second, generates at least 76 signal pulses in total. In this way, the quantitative analysis of milk flowing through the flow line (220) is determined with increased precision.

Each infrared transmitter (121) transmits the infrared ray on and off at a specific frequency. Each of the 10 infrared transmitters (121) has a different frequency. In this way, the frequency of the transmitted infrared ray is detected by the infrared receivers (122). In this way, it is determined by filtering which infrared transmitter (121) the detected infrared ray comes from. Thus, milk quantity measurement is realized with increased sensitivity.

In a possible embodiment of the invention, the infrared transmitters (121) are tunable transmitters capable of 15 degrees of propagation. Furthermore, the distances of the infrared receiver (122) and the infrared transmitters (121) from each other are configured according to said degree of propagation. In this way, the problem of infrared receivers (122) and infrared transmitters (121) interfering with each other is eliminated.

The milk measuring device (10) comprises a conductivity sensor (131) configured to be at least partially in contact with milk flowing through the flow line (220). Said conductivity sensor (131) measures the conductivity of the milk flowing through the flow line (220). The processor unit (110) comprises an impedance measurement unit (130) for determining the conductivity parameter of the milk. The impedance measurement unit (130) determines the conductivity parameter of milk flowing through the flow line (220) according to a signal received by the processor unit (110) from the conductivity sensor (131). The processor unit (110) is able to detect the presence of milk using the determined conductivity parameter of the milk. As a result of this detection, since the gap between the moment when the milk first enters and the moment when it exits is known and the transition time can also be determined, the transition speed of the milk can be calculated. Based on this, the processor unit (110) determines the pressure of the milk flowing through the flow line (220).

The milk measuring device (10) comprises a heat sensor (141) for measuring the temperature of milk flowing through the flow line (220). Said heat sensor (141) preferably measures the temperature of a heat transmission element, preferably made of a thermally conductive material, which is at least partially in contact with the milk flowing through the flow line (220). The processor unit (110) comprises a temperature measuring unit (140) for determining the heating parameter of the milk. Said temperature measuring unit (140) determines the temperature of milk flowing through the flow line (220) according to a signal received by the processor unit (110) from the heat sensor (141). In a possible embodiment, the temperature of the milk flowing through the flow line (220) determined by the heat measurement unit (140) is used by the impedance measurement unit (130) to determine the conductivity parameter of the milk. In this way, the effect of the temperature of the milk on its conductivity is taken into account and the conductivity of the milk is determined with improved accuracy.

The milk measuring device (10) comprises a light source (151) directed towards the flow line (220) for emitting visible light wavelength. Said light source (151) is preferably positioned on the first circuit board (101). The light source (151) may be any light-emitting element known in the art. In the preferred embodiment, the light source (151) is an RGB LED. The milk measuring device (10) comprises an optical sensor (152) provided opposite the light source (151) for detecting light emitted by the light source (151). Said optical sensor (152) generates a signal according to the light detected. The optical sensor (152) is a photosensor sensitive to the wavelength of light known in the art. The processor unit (110) comprises a color measurement unit (150) for determining a color parameter of the milk flowing through the flow line (220). Said color measurement unit (150) determines the color of the milk flowing through the flow line (220) according to the signal received by the processor unit (110) from the optical sensor (152). In this way, the amount of blood contained in the milk flowing through the flow line (220) can be determined. In addition, milk containing blood can be prevented from mixing with other milk.

In a possible embodiment of the invention, the milk measuring device (10) comprises a pressure sensor (161) for measuring the pressure of milk passing through the flow line (220). The milk measuring device (10) further comprises a pressure transmission element for providing a relation between said pressure sensor (161) and the milk flowing through the flow line (220). Said pressure transmission element is a sealed silicone membrane or any suitable pressure transfer material known in the art, which is provided on the flow line (220). The pressure sensor (161) measures the pressure applied by the milk flowing through the flow line (220) on the pressure transmission element. The processor unit (110) comprises a pressure measuring unit (160) for determining the pressure parameter of milk flowing through the flow line (220). Said pressure measurement unit (160) determines the pressure of the milk flowing through the flow line (220) according to the signal received by the processor unit (110) from the pressure sensor (161). Thus, by means of the pressure measurement information, the vacuum and pulsation test of the milking system is performed. With the vacuum and pulsation test, problems in the milking system are detected. In addition, the pressure information is also used in the measurement calibration of the milk measuring device (10). In this way, the accuracy of the parameters measured by the milk measuring device (10) is increased.

In a possible embodiment of the invention, the first circuit board (101) and the second circuit board (102) comprise input and output pins (103) for exchanging data with each other. Said input and output pins (103) enable the first circuit board (101) and the second circuit board (102) to be electrically connected.

In a possible embodiment of the invention, the milk measuring device (10) comprises a user interface (170). Said user interface (170) enables the user to read out parameters set by the processor unit (110).

In a possible embodiment of the invention, the milk measuring device (10) comprises a memory unit (180). Said memory unit (180) enables the parameters determined by the processor unit (110) to be stored in relation to the milking animal information. The memory unit (180) may be an internal or external storage element.

In a possible embodiment of the invention, the milk measuring device (10) comprises a communication unit (190). Said communication unit (190) enables external industrial devices to communicate with the milk measuring device (10). The communication unit (190) may have communication interfaces known in the art, such as RS485, RS422, or CAN.

In a possible embodiment of the invention, the milk measuring device (10) comprises a power unit (200) for supplying energy to the electrical components therein. Said power unit (200) may be a component providing electrical energy, such as a battery or an adapter. The power unit (200) may be an industrial power module with 9V-36V high voltage, low voltage, and circuit protection. The power unit (200) protects the circuit boards in the milk measuring device (10) from damage to the electronic board in case the DC voltage input of the circuit boards is connected in the opposite direction.

In a possible embodiment of the invention, the milk measuring device (10) comprises a button. Said button performs a reset so that the data of the animal whose data is taken is not confused with the data of the animal awaiting measurement. The button is preferably provided on the user interface (170).

In an operation scenario of the flow measurement unit (120), there are 2×5 infrared transmitters (121) and infrared receivers (122) arranged opposite each other with the flow line (220) between them. These infrared transmitters (121) and infrared receivers (122) detect the quantity of milk and the air content. Infrared rays from the infrared transmitter (121) reach the infrared receiver (122). These infrared rays generate a value according to the perception of the infrared receiver (122). When these infrared rays pass through only air and only milk, they give different values. When milk and air are together, the infrared rays will give a value between the values when they pass through the air only and milk only, depending on the amount of air in the milk, and thus the amount of air in the milk can be determined. For example, in an air-only flow, the reading from these infrared receivers (122) will be maximum—because the diffraction of the ray will be minimal—. In a flow with only milk, the reading from these infrared receivers (122) will be minimum, since the diffraction of the ray will be maximum. In a flow with a mixture of air and milk, the reading will be between a maximum and a minimum, since the ray refraction will vary according to the proportion of air in the milk. From this, the amount of air in the milk can be determined according to the reading. These infrared receivers (122) will be able to do this not only in opposite directions but also in diagonal directions, allowing the amount of air in the milk to be determined even more precisely.

In an example operation scenario of the color measurement unit (150), blood in the milk is detected by means of an optical sensor (152) in the milk measuring device (10). Opposite this optical sensor (152) is 1 light source (151) (RGB LED). This RGB LED sends a white light toward the optical sensor (152). White light is emitted because it is a mixture of the primary colors red, blue, and green. Each color has a different wavelength and their combination in different proportions creates different types of colors. When the white light hits the blood in the milk, only the red color will be reflected to the optical sensor (152) as the other colors will be absorbed before reaching the optical sensor (152). In this way, the presence of blood in the milk can be determined by the processor unit (110).

In an exemplary operation scenario of the impedance measurement unit; stainless steel rods are positioned opposite each other at the inlet opening (210) where milk enters the milk measuring device (10) and at the outlet opening (230) where milk exits. When the milk comes into contact with these steel bars, a change occurs in the electrical signal sent to the steel bars. The frequency change of the electrical change is measured. By converting the electrical conductivity into frequency, the following information is obtained from the conductivity values at the inlet and outlet points of the milk;
Vacuum pressure with milk transit speed and conversion from the time between the inlet and outlet speed of milk
From the conductivity value, the amount of air in the milk and the milk fullness rate (Less amount of air means higher conductivity of the milk).
Since the conductivity of the animal's past milk is recorded in the memory unit (180), comments can be made about animal health according to the conductivity change. In addition, according to the conductivity value, it is understood whether the amount of somatic cells in the milk is less or more.

In a possible embodiment of the invention, the processor unit (110) combines the conductivity measurement from the conductivity sensor (131) with the flow measurements from the infrared receivers (122), the color measurements from the optical sensor (152), the heat measurements from the heat sensor (141) and the pressure measurements from the pressure sensor (161) by means of a sensor fusion process. In this way, the amount of milk flowing through the flow line (220) is determined with improved accuracy.

In an example operation scenario of the heat measurement unit (140); the temperature value of the milk is determined by changing the resistance value in the heat sensor (141) used. Since the animal-milk measuring device is matched, the temperature of the animal's previous milk is known through the memory unit (180). According to the temperature increase here, it is understood whether the animal has a fever or whether it has different diseases.

The protection scope of the invention is set out in the appended claims and cannot be strictly limited to what is described in this detailed description for illustrative purposes. It is clear that those skilled in the art can come up with similar embodiments in the light of the foregoing without departing from the main theme of the invention.

### REFERENCE NUMBERS GIVEN IN THE FIGURE

10 Milk measuring device
101 First circuit board
102 Second circuit board
103 Input and output pins
110 Processor unit
120 Flow measurement unit
   121 Infrared transmitter
   122 Infrared receiver
130 Impedance measurement unit
   131 Conductivity sensor
140 Heat measurement unit
   141 Heat sensor
150 Color measurement unit
   151 Light source
   152 Optical sensor
160 Pressure measurement unit
   161 Pressure sensor
170 User interface
180 Memory unit
190 Communication unit
200 Power unit
210 Inlet opening
220 Flow line
230 Output opening

1. A milk measuring device for determining at least one parameter of milk flowing through a flow line provided between an inlet opening and an outlet opening, wherein said flow line comprises:
a first circuit board and a second circuit board configured to remain there between;
said first circuit board comprises a plurality of infrared transmitters directed towards said flow line for emitting infrared ray;
said second circuit board comprises a plurality of infrared receivers directed towards said first circuit board for detecting the infrared ray emitted by said infrared transmitters and generating a signal according to the detected infrared ray;
the infrared ray emitted by each infrared transmitter is detected by at least two infrared receivers that comprise a processor unit for ensuring simultaneous operation of the infrared transmitter and the infrared receivers; and
the processor unit is configured to determine at least one flow parameter of the milk flowing through the flow line according to the signal generated by the infrared receivers.

2. A milk measuring device according to claim 1, wherein the infrared transmitters provided on the first circuit board and the infrared receivers provided on the second circuit board are arranged in equal numbers and opposite each other.

3. A milk measuring device according to claim 2, wherein the infrared transmitters provided on the first circuit board and the infrared receivers provided on the second circuit board are arranged in two rows of five.

4. A milk measuring device according to claim 2, wherein the infrared ray emitted by each infrared transmitter is detected by the infrared receiver provided opposite thereto and at least five infrared receivers closest to the infrared receiver provided opposite thereto.

5. A milk measuring device according to claim 1, further comprising a conductivity sensor for measuring the conductivity of milk, configured to be at least partially in contact with milk flowing through the flow line; wherein the processor unit is configured to determine at least one conductivity parameter of milk flowing through the flow line according to the signal it receives from said conductivity sensor.

6. A milk measuring device according to claim 1, further comprising a heat sensor for measuring the temperature of milk flowing through the flow line; wherein the processor unit is configured to determine at least one heat parameter of milk flowing through the flow line according to a signal it receives from said heat sensor.

7. A milk measuring device according to claim 1, wherein the first circuit board comprises a light source directed towards the flow line for emitting visible light wavelength; the second circuit board comprises an optical sensor directed towards the first circuit board for detecting the light emitted by said light source and generating a signal according to the detected light; wherein the processor unit is configured to determine at least one color parameter of milk flowing through the flow line according to the signal it receives from the said optical sensor.

8. A milk measuring device according to claim 1, further comprising a pressure sensor for measuring the pressure of milk flowing through the flow line and a pressure transmission element for providing a relation between said pressure sensor and the milk flowing through the flow line; wherein the processor unit is configured to determine at least one pressure parameter of milk flowing through the flow line according to a pressure signal it receives from the pressure sensor.

9. A milk measuring device according to claim 1, characterized wherein said flow line is made of infrared ray transmitting material.

10. A milk measuring device according to claim 1, wherein at least part of said flow line is configured to transmit visible light wavelength.

11. A milk measuring device according to claim 1, further comprising a memory unit; wherein the processor unit is configured to store the determined parameters in the said memory unit by associating it with the milked animal information.

12. A milk measuring device according to claim 1, further comprising a communication unit for enabling external industrial devices to communicate with the milk measuring device; wherein said communication unit is associated with the processor unit.

13. A milk measuring device according to claim 1, further comprising a user interface for enabling the user to read out parameters determined by the processor unit.
